(19) United States
(12) Patent Application Publication
(10) Pub. No.: US 2025/0073504 A1
(43) Pub. Date: Mar. 6, 2025

(54) TECHNIQUES FOR SKULL ABERRATION CORRECTION FOR TRANSCRANIAL FOCUSED ULTRASOUND

(71) Applicant: Sanmai Technologies, PBC, Sunnyvale, CA (US)

(72) Inventors: Christopher DAFT, Tucson, AZ (US); Ashish PARIKH, Los Altos, CA (US); Bicheng WU, Palo Alto, CA (US)

(21) Appl. No.: 18/816,823

(22) Filed: Aug. 27, 2024

Related U.S. Application Data
(60) Provisional application No. 63/580,071, filed on Sep. 1, 2023.

(51) Int. Cl. A61N 7/00

(52) U.S. Cl. CPC A61N 7/00; A61N 2007/0026

(57) ABSTRACT

A transcranial Focused Ultrasound (tFUS) system uses a neural network to correct skull aberrations and maximizes the transmission of ultrasound waves through the skull. A method using supervised learning generates aberration correction parameters to be used by the receiver and transmitter of the tFUS system. A method utilizing these aberration correction parameters operating on the tFUS system maximizes the coherence of ultrasound waves passing through the skull. The method maximizes the amount of power transmitted through the skull, given a fixed maximum pressure (for example, determined by regulatory requirements).

### CROSS-REFERENCE TO RELATED APPLICATIONS

This application claims the priority benefit of U.S. Provisional Patent Application No. 63/580,071 filed Sep. 1, 2023 and entitled “TECHNIQUES FOR SKULL ABERRATION CORRECTION FOR TRANSCRANIAL FOCUSED ULTRASOUND,” the contents of which are incorporated by reference herein.

### TECHNICAL FIELD

This application relates to trans-cranial focused ultrasound systems (tFUS), particularly for correcting for skull aberrations.

### BACKGROUND

Transcranial focused ultrasound (tFUS) is an emerging neuromodulation technology in which low-intensity ultrasound is non-invasively transmitted into shallow or deep brain regions. tFUS has the advantage of better spatial resolution than other non-pharmaceutical interventions such as transcranial magnetic stimulation (TMS) or the electrical techniques TDCS (transcranial direct current stimulation) and TACS (transcranial alternating current stimulation). The human skull varies in thickness, density, and sound speed and thus presents a severe challenge to techniques requiring focusing ultrasound within the brain.

FIG. 1A is a prior-art example of a tFUS system consisting of a transducer 110 and an example skull fragment 120. The skull fragment 120 is of varying width (not to scale), illustrated by width 1 (W1) and width 2 (W2). The effects of the skull variations are studied at two (focal) points within the brain and represented as locations 125-01 and 125-02. FIG. 1B is a prior art example waveform at the output of the transducer 110. It shows the pressure of the incident waveform on the Y-axis and time on the X-axis. The incident waveform starts at time 0, and the peak and low amplitudes are shown as P1 and P0, respectively. FIG. 1C and FIG. 1D are prior art example waveforms observed at 125-01 and 125-02. FIG. 1C corresponds to location 125-01; the waveform is delayed and starts at time T01. FIG. 1C also illustrates the attenuation. The incident waveform is followed by a spurious wave caused by aberrations (multiple reflections resulting in replicas of the incident waveform with various delays and amplitudes) in the skull, illustrated as 130 in the figure. FIG. 1D corresponds to location 125-02 and shows that the delay differs from location 125-01 and the incident waveform starting at T02. The spurious wave 130 at 125-02 is further delayed. The varying thickness and density of the skull cause non-uniform refraction, attenuation, and aberrations to the incident ultrasound waves. Similarly, the reflected waveforms are also affected by skull variations. There is a need to improve the efficacy of the ultrasound wave used for tFUS. An ideal solution would be to create a sound field in the brain that can be as easily focused and steered as with soft tissue imaging, such as is done routinely in diagnostic ultrasound of the abdomen.

### BRIEF DESCRIPTION OF THE DRAWINGS

FIG. 1A is a prior-art example of a tFUS system. FIGS. 1B, 1C, and 1D are prior-art examples of a transmitted ultrasound waveform as it travels through a human skull.

FIG. 2 is an exemplary tFUS system 200 that uses skull aberration correction parameters to maximize the transmission of ultrasound waves through the skull.

FIG. 3 shows an exemplary block diagram of a computer system 300 that trains a neural network to compensate for skull aberrations.

FIG. 4 shows an exemplary diagram of a supervised learning system 400 that generates transmit and receive skull aberration correction parameters for skull fragments.

FIG. 5 is a flowchart showing a process performed by computer system 300 to generate skull aberration correction parameters according to an embodiment.

FIG. 6 is a flowchart showing a process performed by system 200 to correct skull aberrations and optimize ultrasonic energy transmission through the skull according to an embodiment.

FIG. 7 shows an exemplary waveform 700 illustrating skull aberration correction parameters.

### DETAILED DESCRIPTION

A transcranial Focused Ultrasound (tFUS) system uses a neural network to correct skull aberrations and maximizes the transmission of ultrasound waves through the skull. A method using supervised learning generates aberration correction parameters to be used by the receiver and transmitter of the tFUS system. A method utilizing these aberration correction parameters operating on the tFUS system maximizes the coherence of ultrasound waves as they exit the skull. The method also maximizes the power transmitted through the skull, given a fixed maximum pressure. Regulatory requirements may determine the maximum pressure that the probe can emit.

Here we disclose a method for using a neural network to learn a useful way to alter beam formation coefficients, transmit waveforms, and receive processing when traditional beam formation produces poor results. Examples of where such an approach finds applications are in optimizing the stimulation focus in tFUS and for the guidance of the stimulation beam using pulse-echo ultrasound techniques.

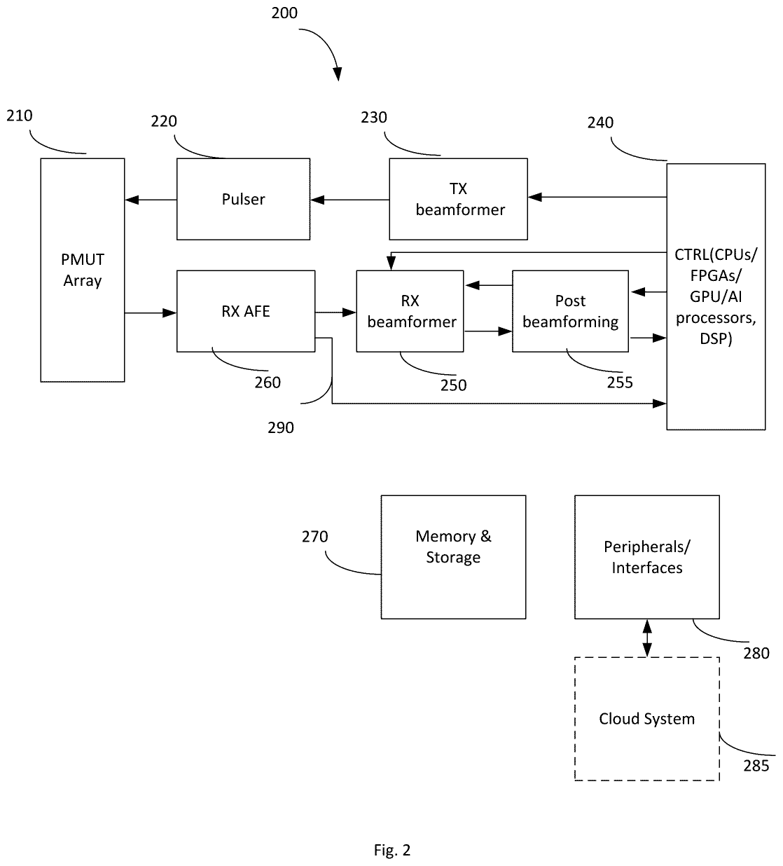

FIG. 2 is an exemplary tFUS system 200 that uses skull aberration correction parameters to maximize the transmission of ultrasound waves through the skull. tFUS system consists of an array of transducers 210. The example in the figure shows a single transducer array, more than one transducer array can be used. A transducer converts an electrical signal into mechanical and mechanical vibrations into an electrical signal. Array 210 includes ultrasound transmitters and ultrasound receivers. Array 210 receives electrical signals from Pulser 210 and converts them into ultrasound signals to be transmitted into the brain. The skull and brain generate a reflected ultrasound signal received by array 210, converted into an electrical signal, and sent to RX AFE 260. Array 210 may use PMUT (Piezoelectric Micromachined Ultrasonic Transducers), but other implementations (e.g., Capacitive Micromachined Ultrasonic Transducers or bulk piezoelectric transducers) are possible. PMUT array 210 is placed on the skull, and care is taken to ensure proper contact is made so that the ultrasound waves couple well to the scalp. PMUT array 210 can be configured to consist of various numbers of transmitters and receivers; individual elements may be used as transmitters only, receivers only, or for both operations. PMUT array 210 can be placed on the skull in one location, or more than one array may be used to stimulate spatially diverse volumes. Each array can steer and focus the ultrasound within a roughly conical volume. In one example, array 210 is connected to a multi-channel ultrasound transmitter capable of producing stimulation and guidance waveforms. The stimulation waveform can be long tone bursts to optimize the physiological effect, while the guidance waveforms can be short, usually 1%, 1, or a few cycles, to optimize the axial resolution. The guidance transmit waveform and the stimulation waveform may be adjusted via the aberration correction computation. Similarly, during the ultrasound reception part of the guidance system, the aberration correction computation informs the beam formation delay and other processing such as the impulse response of a filter applied to the signal. In an implementation, array 210 consists of a multi-element transducer. A portion of this transducer may be specialized for optimal stimulation frequency, while another is optimized for resolution to produce the best guidance. The guidance part of the transducer may be split into transmit or receive portions or used for both transmit and receive. Alternatively, transducer elements may be used both for stimulation and guidance functions.

System 200 receives skull aberration correction parameters (Amplitude correction Â, aberration delay [symbol] and [ [symbol] ] amplitude \dot{a} & timing “\hat{d}” of the additional negative pulse(s); described in more detail later) using peripherals/interfaces 280 and stored in memory/storage 270 by control 240. Peripherals/interfaces 280 can include wired (USB) and wireless interfaces (WiFi, Bluetooth, etc.) for updating system 200 with neural network weights and other control parameters. Peripherals/interfaces 280 can include power-related interfaces (buttons, charging ports, etc.). Peripherals/interfaces 280 can consist of other peripherals such as LEDs, speakers, and vibration devices to provide feedback to users. Feedback can be about the placement of array 210 or the system's status. Memory and storage 270 can consist of volatile and non-volatile memory and storage (HDD/SSD) and are used to store the aberration correction parameters. Memory & Storage 270 stores programs, instructions, and data needed to operate CTRL 240 (control). CTRL 240 controls the overall operation of system 200. It may include one or more FPGAs (Field Programmable Gate Arrays), GPUs (Graphics Processing Units), DSPs (Digital Signal Processors), CPUs (Central Processing Units), Neural Network processors, or AI processors. CTRL 240 implements an inference NN (neural network) to apply corrections to the transmitted and received ultrasound waveforms.

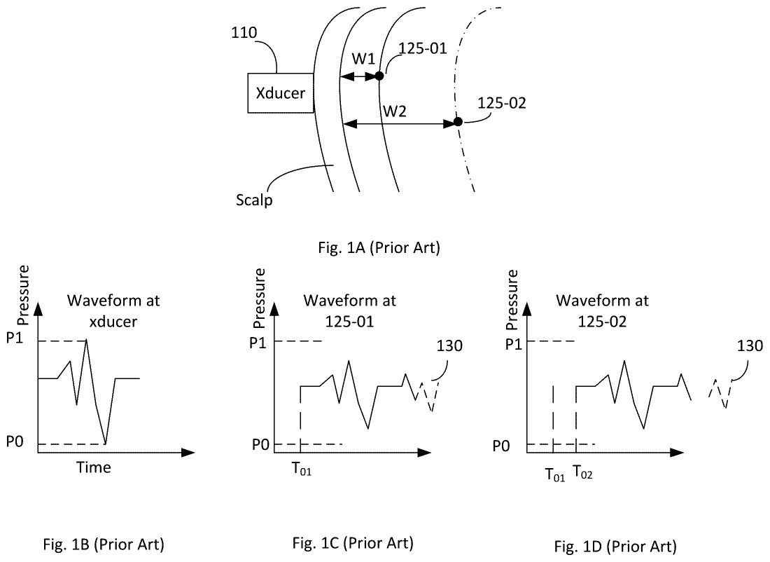

On the transmit side, the skull aberration correction parameters include changing the transmit signal's time from the simple geometrical focusing value and an amplitude correction. Additional corrections include transmitting additional pulses to counteract reflections created by interfaces within or at the surfaces of the skull (such as 130 shown in FIG. 1C and FIG. 1D). Correction parameters include the timing and amplitude of these additional pulses. System 200 computes a transmit waveform that may work cooperatively with reflections from the skull to optimize ultrasonic energy transmission through the skull. Based on the placement of PMUT array 210, it makes the necessary corrections (using the NN-computed skull aberration parameters) to maximize waveforms' efficacy (power transmitted) while ensuring it is safe for the subject and meets the regulatory requirements. In an embodiment, reflected US (Ultrasound) waves parameters such as echo time, echo strength, scattering, absorption loss estimates, blood flow speed and volume measurement, tissue strain, pulsatility, etc., are used to modify the phase, amplitude, etc., of the transmitted stimulation waveforms and further improve the energy transmission through the skull.

CTRL 240 has several functions. It includes an inference neural network that takes as its input channel data 290 from the received elements and computes the alterations to the beamformer timing and the transmit and receive gain. These alterations are provided to the TX beamformer 230 and RX beamformer 250.

A single application-specific integrated circuit (ASIC) may provide the functions of the pulser 220, RX AFE 260, TX beamformer 230, and RX beamformer 250. Alternatively, these functions may be implemented in separate integrated circuits. TX beamformer 230 controls the transmitted ultrasound signals' steering direction, focal depth, and pulse pattern. Geometrical considerations are only part of the computation of channel delay and amplitude values; corrections from the neural network within CTRL 240 are also considered. Pulser 220 receives this information and generates the necessary signals for array 210. The echoes or received signals from array 210 are sent to RX AFE 260. RX AFE 260 provides the necessary amplification, filtering, signal processing, etc., for the signal received from array 210. The signal from RX AFE 260 is received by RX beamformer 250, which may have both analog and digital processing sections. RX beamformer 250 creates the received beam using delay values and channel amplitudes, which are adjusted to compensate for skull aberrations. CTRL 240 provides the necessary amplitude and other beamforming corrections to RX beamformer 250. CTRL 240 may also provide additional instructions to RX beamformer 250 for further processing. These instructions may also include alterations to filter parameters used in post-beam-formation block 255.

Using Peripheral/interfaces 280 (such as wired or wireless interfaces), System 200 transmits data such as transducer location (on the skull), skull aberration correction parameters used, transmit signal parameters (phase, amplitude, stimulation, and guidance waveform details), and received signal details (echo time, echo strength, scattering, absorption loss estimates, blood flow speed and volume measurement, tissue strain, pulsatility, etc.) to a cloud system 285. System 200 transmits these details to the cloud system 285 after every single use or periodically. Cloud system 285 is connected to many Systems 200. From the data gathered from many Systems 200, the Cloud system 285 runs correction algorithms (such as supervised learning) to improve the skull aberration correction and other parameters. These updated parameters are sent to System 200.

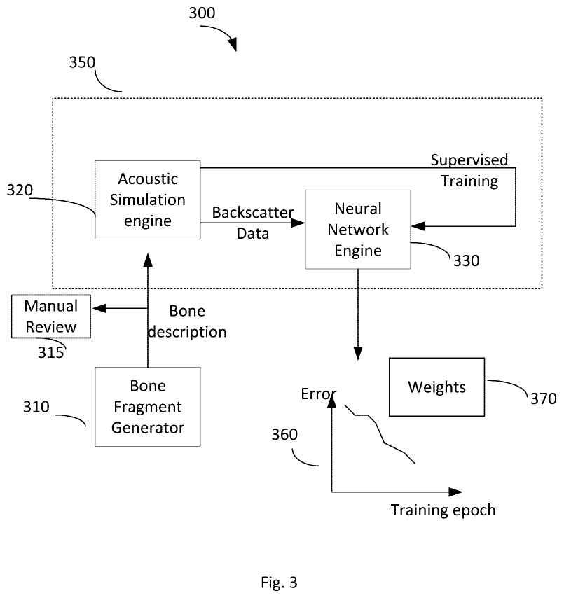

FIG. 3 shows an exemplary block diagram of a computer system 300 that trains a neural network to compensate for skull aberrations. System 300 includes a compute server 350 comprising an acoustic simulation engine 320 and a neural network engine 330. The computer system 300 may either be on-premise or in the cloud. System 300 contains a bone fragment generator 310 that generates anatomically realistic, random, in-silico models of various human skull fragments of varying thickness, curvature, sound speed, and density.

Measurements on human skulls may be used to provide parameter distributions of geometric and mechanical properties of the simulated bone fragments. In-vivo data may be collected using X-ray computed tomography or magnetic resonance imaging. For in-vitro skull fragments, micro-CT systems such as those manufactured by Perkin-Elmer can provide exquisite detail in resolving mechanical properties.

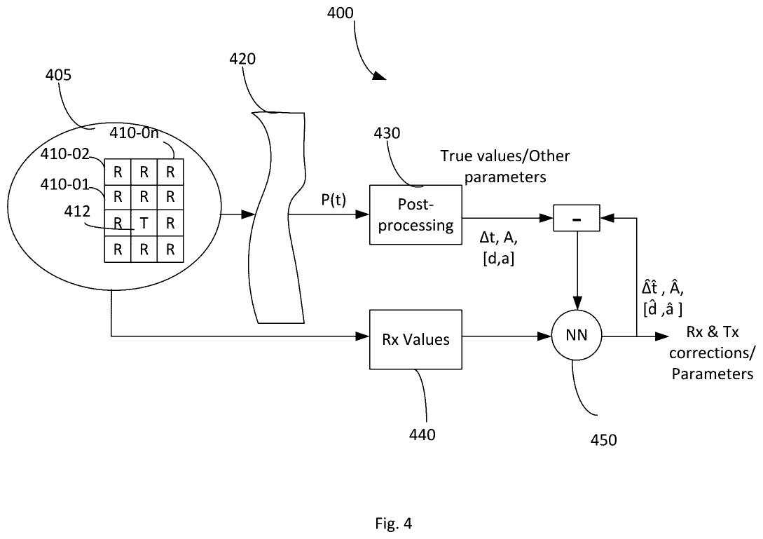

A large number of bone fragments generated by unit 310 are submitted to the acoustic simulation engine 320, potentially after a manual or automatic review 315. The review ensures diversity in the characteristics of bone fragments generated as well as checking for realistic anatomical behavior. Important characteristics include internal layering, thickness, curvature, sound speed, and density. Acoustic simulation engine 320 uses a pseudo-spectral simulator such as k-wave, a finite-element model such as ANSYS, or another type of simulation program. Acoustic simulation engine 320 generates the backscatter data fed into the Neural network 330. The neural network 330 is trained in a supervised manner until its errors are sufficiently low to produce good results (360). During training, the neural network weights (370) are constantly updated. Though not explicitly shown, system 300 includes the necessary hardware, such as CPU, Neural Network processors, memory, storage, etc. Details of how the supervised training is accomplished are shown in FIG. 4.

FIG. 4 shows an exemplary diagram of a supervised learning system 400 that generates transmit and receive aberration correction parameters for skull fragments. System 400 may run on computer architecture 300 described previously. Using a pseudospectral (or other) simulator 320, the acoustic effects of sound speed (c[x,y,z]) and density (p[x,y,z]) at each point of bone fragment 420 is simulated. Backscattered and transmitted pressures are computed using spatial distributions of material parameters. The simulation results such as pressure distributions can generate aberration correction parameters. System 400 comprises in-silico transducer 405, in-silico bone fragment 420, soft tissue to represent the scalp and portions of the brain, post-processing block 430, and NN 450 (neural network). Bone fragment 420 represents the soft tissue of the scalp as well as portions of the skull and the brain. Bone fragment 420 must be larger than the transducer array with (n×m) elements. In this example, a group of elements within the array 405, such as a (4×3) group, i.e., with four rows and three columns, are simulated. Other group configurations are possible; for example, a group in the array 405 can comprise a (5×5) set of elements. One of the transducers (“k”) in array 405, ideally in the center of the group, is configured to be a transmitter (labeled 412), and the rest are configured to be receivers (labeled 410-01, 410-02, . . . 410-nn). Pressure waveform Pₜ(k,t) is generated by transducer element 412 and ultrasound propagates to bone fragment 420. Fragment 420 scatters some of the incident ultrasound back towards the array of receivers, while a portion of the ultrasound is transmitted through the bone. Backscattered pressures incident on transducers in group 410 are converted into Vᵣ(m,n,t), voltages received at each ultrasound receiver 410-01, . . . , 410-0n. The voltage Vᵣ(m,n,t) and other received values (pressure, transmitter location “m”) are processed, digitized, and captured in RX Values 440.

The transmitted pressure P(k,t) at a single point beyond the skull fragment 420 is also captured. Each skull fragment's transmit (TX) and receive (RX) parameters are sent to the NN 450 for training. The backscattered data is provided to the NN at its inputs, while the transmitted data is used to create the ideal parameters (Δt, A, [d, a]). The error values supplied to the NN, enabling supervised learning, are created by comparing the ideal values found from the transmitted ultrasound (Δt, A, [d, a]) from the predicted values computed by the NN, and generating an error signal therefrom. The weights and biases of the NN are trained via backpropagation, or another machine learning algorithm.

Note that the NN only ever has access to the backscattered data Vᵣ(m,n,t). It never sees the sound field beyond the skull Pₜ(k,t), which is only used by the post-processing block in the training system to create the ideal parameters.

During inference, the NN has access to the backscattered data from the subject's skull, and it must create the correction parameters solely from that data since there is no access to Pₜ(k,t) in-vivo.

The above process is repeated with different bone fragments over a large training data set. A separate validation data set is also computed, which is never shown to the training system. It is used solely after the weights are frozen to assess the neural network's performance.

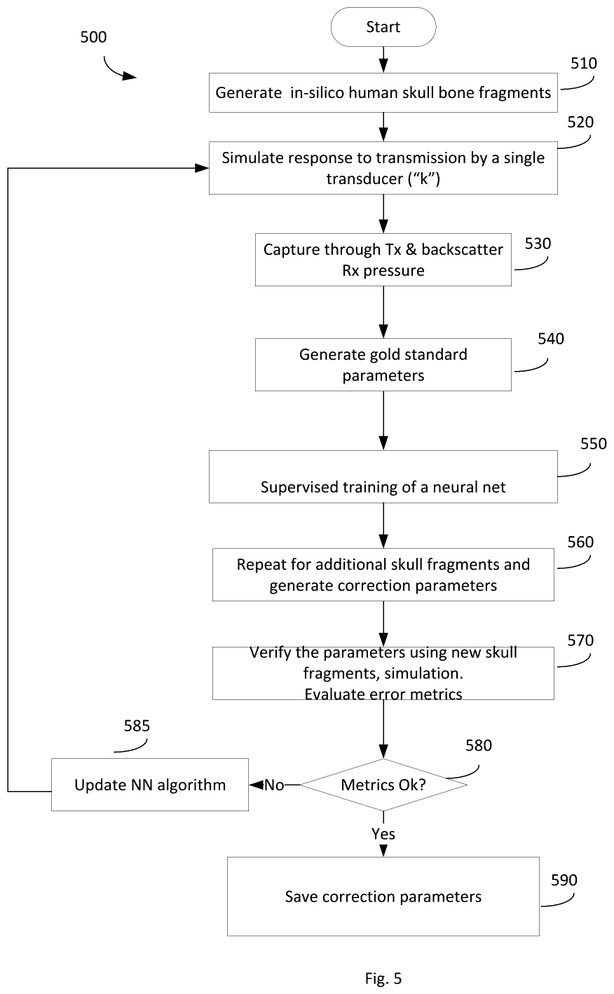

FIG. 5 is a flowchart showing a process performed by computer system 300 to generate skull aberration correction parameters according to an embodiment. Referring to FIG. 5, in operation 510, realistic, random, in-silico bone descriptions of various human skull fragments of varying layering, thickness, curvature, sound speed, and density are created. For example, bone fragment generator 310 can create in-silico models. Alternatively, in addition to this, in-vivo or in-vitro measurements of human skulls, as mentioned above, may be used.

In operation 520, the response of the bone fragment 420 to sound pressure from a single transmitter “k” 412 is simulated. Transmitted pressure Pₜ(k,t) is the portion of the pressure field generated by transmitter 412 which passes through known bone fragment 420.

At operation 530, transmitted pressure Pₜ(k,t) and other parameters at a single point beyond the bone fragment 420 is captured. Additionally, the backscattered pressure responses to the bone fragment 420 are captured by the “m×n” adjacent transducers 410. Element pressure captured by adjacent receivers 410 is converted into Vᵣ(m,n,t), the voltages received at each ultrasound transducer 410-01, . . . , 410-0n. Pₜ(m,t) and Vᵣ(m,n,t) are saved by system 300.

At operation 540, system 300 post processes 430 the saved Pₜ(m,t) to generate gold standard or true aberration correction parameters. At operation 550, Voltages Vᵣ(m,n,t) (saved as RX values 440) are used for the input for supervised training of neural net 450, where the error comprises the differences between the network output parameters and the gold standard parameters.

At operation 560, system 300 performs operations 520 to 550 for numerous additional human skull fragments generated at operation 510. The neural network t and biases are fixed after processing all the human skull fragments, computing the NN estimation errors, and adjusting weights through backpropagation or another training algorithm. In some embodiments, the training set may be used several times for NN training.

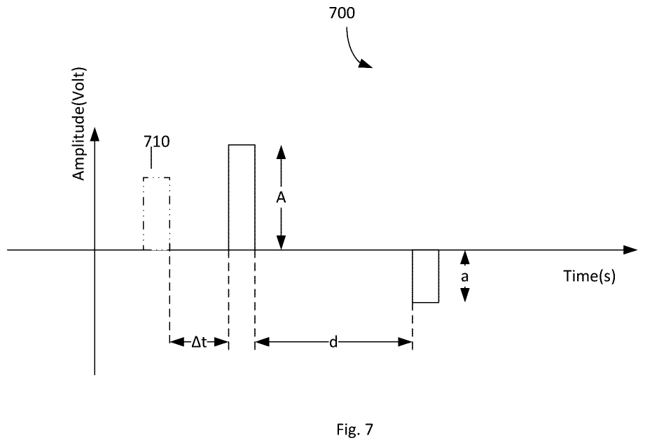

FIG. 7 shows an exemplary waveform 700 illustrating one simple set of four skull aberration correction parameters. Referring to FIG. 7, pulse 710 is a possible original pulse to be transmitted by the transducer. The incident waveform is subject to aberrating delay and loss of signal amplitude. This can be corrected by adding an aberrating delay Δt. The amplitude loss in the skull can be compensated by transmitting the signal with compensated amplitude A. The incident waveform is also subject to spurious waves from reflections within or at the surfaces of the skull. Spurious waves can be compensated by sending an additional negative pulse of amplitude “a” and delayed by “d.” The aberrating delay Δt can also be compensated within the receiver. The applied time delay in the receiver will be opposite to the transmit correction. The received data can be multiplied by “A” to compensate for the amplitude loss. The spurious wave effects can be canceled by convolving with a time-reversed version of FIG. 7's transmit signal Vₜ with an additional negative pulse [a,d]. For bone fragment characteristics, such as thickness, density, etc., final correction parameters are computed as (Â, [symbol] , [ [symbol] ]).

Referring back to FIG. 5, at operation 570, system 300 verifies parameters (Â, [symbol] , [symbol] ) and evaluates the error metrics. A new test set of skull fragments is prepared. The new test set is not used for training the NN 450, i.e., not seen by NN 450 before. The new test set meets constraints such as curvature, bone mechanical properties, and layer thicknesses. For verification, the network adaption is switched off, and each bone fragment is provided to the NN 450 to estimate [Δtₜ, Aₜ] for the test set. Error metrics are calculated as ( [symbol] −Δtₜ)² and (Â−Aₜ)².

A second way of verifying parameters is (Â, [symbol] , [ [symbol] ]) includes simulating and capturing Pₜ(m,t) and comparing the position and amplitude of the corrected pressure with the expected values (i.e., ideal non-skull pressure).

At operation 580, system 300 evaluates error metrics calculated at operation 570. If system 300 determines that metrics are within limits, system 300 continues to operation 590. If error metrics are not within limits, system 300 continues to operation 585.

At operation 585, the algorithm used by NN 450 is updated and system 300 proceeds to operation 520.

At operation 590, system 300 saves correction parameters (Â, [symbol] , [ [symbol] ]) to be used by other systems. For example, skull correction parameters (Â, [symbol] , [ [symbol] ]) are used by system 200.

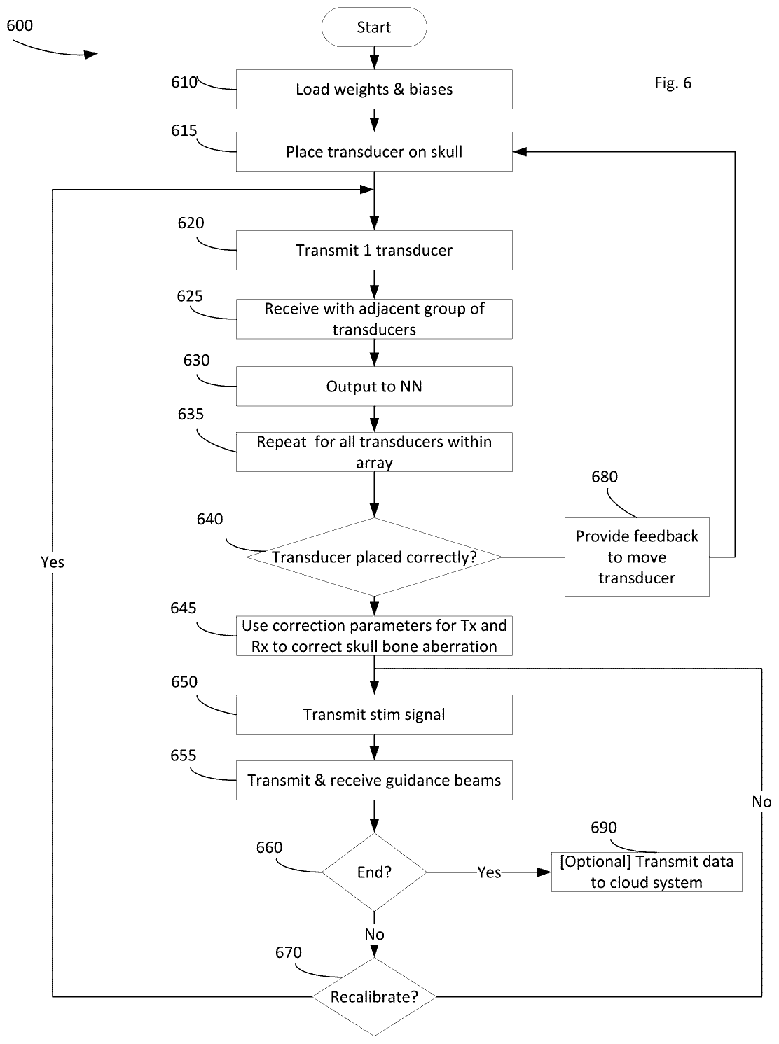

FIG. 6 is a flowchart showing a process performed by system 200 to correct skull aberrations and optimize ultrasonic energy transmission through the skull according to an embodiment. Referring to FIG. 6, at operation 610, system 200 loads neural network weights and biases, geometrical beam formation coefficients, and other data. For example, the weights and biases generated by the flowchart shown in FIG. 5 are loaded into Memory & Storage 270.

At operation 615, transducer array 210 is placed at an appropriate location on the skull of the subject. PMUT array 210 can be placed on the skull in one location, or more than one array may be used to stimulate spatially diverse volumes.

At operation 620, system 200 transmits using a single transducer within transducer array 210. At operation 625, the backscattered US signal is received using a group of transducers adjacent to the single transmit transducer. The US signals are converted into echo waveform Vᵣ(n, t), which represents the received voltage change across time (t) for the receiver (n).

At operation 630, data (received data from the group of transducers) is sent to the neural network (NN) operating in CTRL 240. The neural network can determine the skull properties (thickness, mechanical properties) from the received data. This operation is not compute-intensive.

At Operation 635, Operation 620, Operation 625, and Operation 630 are repeated for all transducer elements with the transducer array 210; i.e., a different transducer is selected for transmission, and the adjacent group captures the echoed US signal. The operations are repeated until all elements within the array 210 have been transmitted, and the corresponding received data is captured by the group of adjacent transducers. In an embodiment, when multiple arrays 210 are placed on the subject's skull Operations 620, 625, and 630 can be performed simultaneously across each transducer array.

At operation 640, system 200 determines if transducer 210 is placed correctly. System 200 determines this by evaluating the backscattered signal. For example, if there is poor acoustic contact, the backscattered signals will have characteristics recognizable by a machine learning system. Backscattered data may also be used to confirm locations of target structures within the brain, a indicating correct placement by the operator. System 200 may determine that transducer array 210 is not properly contacting the human skull (air gap). System 200 may determine that transducer array 210 is optimally placed. If System 200 determines that the transducer array 210 is placed correctly, it proceeds to operation 645; otherwise, System 200 proceeds to operation 680.

At operation 680, system 200 provides feedback to move transducer array 210. System 200 uses Peripherals/Interfaces 280 to give the instructions. It may use a voice command. It may provide visual clues using LEDs. In one embodiment, it provides this instruction via an accompanying App operating on a smartphone, tablet, viewing screen, etc. The instruction may provide details about the direction and distance transducer 210 has to move.

At operation 645, correction parameters (Â, [symbol] , [ [symbol] ]) computed by the NN from backscattered data are used to correct the transmit and receive waveforms for any skull aberrations. Corrections to the amplitude and delay parameters are loaded into TX beamformer 230 and RX beamformer 250. Transmit waveforms are generated by System 200. CTRL 240 generates RX filter coefficients etc. and sends them to the post beamforming 250.

At operation 650, System 200 generates and transmits stimulation signals or waveforms. The stimulation waveform can be long tone bursts to optimize the physiological effect and are corrected for skull aberrations.

At operation 655, System 200 transmits and receives guidance waveforms. The transmit waveform is adjusted for aberration correction. CTRL 240 provides the beam formation aberration correction and other filter corrections to beam forming 250 and post beamforming 255.

At operation 660, System 200 determines if it is the end of the session or treatment. If System 200 determines that it is the end, it proceeds to operation 690; otherwise, System 200 proceeds to operation 670.

At operation 670, System 200 determines if recalibration (that is, a fresh computation of aberration parameters) is required. Recalibration may be required due to the subject moving his/her head causing a a shift in the transducer array 210 position. It may also be performed periodically during the treatment regardless of subject motion. If System determines that recalibration is required it proceeds to Operation 620 otherwise it proceeds to operation 650.

What is claimed is:

1. A method for generating skull aberration correction parameters comprising:
generating a plurality of in-silico skull bone fragments;
generating, using an acoustic simulator, a plurality of simulated acoustic signals that are applied to the plurality of in-silico skull bone fragments;
capturing, using the acoustic simulator, a plurality of backscattered element responses to the plurality of simulated acoustic signals; and
generating, based on the plurality of backscattered responses, skull aberration correction parameters for at least one element of a transcranial ultrasound transducer.

2. The method of claim 1, wherein the plurality of backscattered responses comprise pressures computed based on material parameters of the plurality of in-silico skull bone fragments.

3. The method of claim 1, wherein generating the skull aberration correction parameters comprise aberration correction parameters based upon one or more aberrations in the in-silico skull bone fragments.

4. The method of claim 1, further comprising:
evaluating, using an acoustic signal applied to a test skull fragment, the correction parameters based upon a test response; and
in response to an error calculated on the test response being an acceptable error, applying the skull aberration correction parameters to a transmitter or a receiver of the least one element of a transcranial ultrasound transducer.

5. The method of claim 1, wherein generating the plurality of in-silico skull bone fragments further comprises generating random skull bone fragment models.

6. The method of claim 1, wherein generating the plurality of in-silico skull bone fragments further comprises varying at least one of a thickness or density of a skull bone fragment model.

7. The method of claim 1, wherein generating the plurality of in-silico skull bone fragments further comprises randomly varying at least one of a thickness or density of a skull bone fragment model.

8. The method of claim 1, wherein generating the skull aberration correction parameters comprises executing a supervised learning process on the plurality of backscattered element responses.

9. The method of claim 8, wherein the supervised learning process generates the skull aberration correction parameters based upon the plurality of backscattered element responses using spatial distributions of material parameters.

10. A method comprising:
receiving a plurality of skull aberration correction parameters;
transmitting, using at least one transcranial ultrasound array transducer, an acoustic signal based on the skull aberration correction parameters;
receiving, using the at least one transcranial ultrasound transducer or at least one transducer element, a backscattered signal in response to the acoustic signal;
calculating a plurality of skull properties based on the backscattered signal;
determining, based on the plurality of skull properties, that the at least one transcranial ultrasound transducer is correctly placed; and
in response to determining that the at least one transcranial ultrasound transducer is correctly placed, transmitting a stimulation signal using the at least one transcranial ultrasound transducer based on the plurality of skull aberration correction parameters.

11. The method of claim 10, wherein transmitting the stimulation signal based on the plurality of skull aberration correction parameters comprises modifying a signal time or an amplitude of the stimulation signal for each transducer array element.

12. The method of claim 10, wherein transmitting the stimulation signal based on the plurality of skull aberration correction parameters comprises transmitting an additional pulse signal to counteract reflections at a surface of a skull indicated by the plurality of skull properties.

13. The method of claim 10 wherein transmitting the stimulation signal based on the plurality of skull aberration correction parameters comprises transmitting long tone bursts from transducer array elements based on the plurality of skull aberration correction parameters.

14. The method of claim 10, wherein transmitting the stimulation signal based on the plurality of skull aberration correction parameters comprises applying a plurality of corrections to the stimulation signal using an inference neural network, wherein the plurality of skull aberration correction parameters are an input to the inference neural network.

15. The method of claim 10, further comprising determining a location of a target structure within a brain based upon the backscattered signal.

16. The method of claim 10, further comprising:
determining, based upon an analysis of the backscattered signal, that the transcranial ultrasound transducer is incorrectly placed; and
generating, based upon the determination that the transcranial ultrasound transducer is incorrectly placed, a feedback signal to relocate the transcranial ultrasound transducer.

17. A transcranial ultrasound system, comprising:
a transcranial ultrasound array transducer;
a processor executing; and
a non-transitory computer-readable medium having stored thereon instructions that, when executed by the processor, cause the processor to perform operations including:
receiving a plurality of skull aberration correction parameters;
transmitting, using the transcranial ultrasound transducer, an acoustic signal based on the skull aberration correction parameters;
receiving, using the transcranial ultrasound transducer, a backscattered signal in response to the acoustic signal;
calculating a plurality of skull properties based on the backscattered signal;
determining, based on the plurality of skull properties, that the transcranial ultrasound transducer is correctly placed; and
in response to determining that the transcranial ultrasound transducer is correctly placed, transmitting a stimulation signal using the transcranial ultrasound transducer based on the plurality of skull aberration correction parameters.

18. A transcranial ultrasound system, comprising:
a first transcranial ultrasound array transducer;
a second transcranial ultrasound transducer;
a processor executing; and
a non-transitory computer-readable medium having stored thereon instructions that, when executed by the processor, cause the processor to perform operations including:
receiving a plurality of skull aberration correction parameters;
transmitting, using the first transcranial ultrasound transducer, an acoustic signal based on the skull aberration correction parameters;
receiving, using the second transcranial ultrasound transducer, a backscattered signal in response to the acoustic signal;
calculating a plurality of skull properties based on the backscattered signal;
determining, based on the plurality of skull properties, that the first transcranial ultrasound transducer is correctly placed; and
in response to determining that the first transcranial ultrasound transducer is correctly placed, transmitting a stimulation signal using the first or second transcranial ultrasound transducer based on the plurality of skull aberration correction parameters.

19. The transcranial ultrasound system of claim 18, wherein transmitting the stimulation signal based on the plurality of skull aberration correction parameters comprises modifying a signal time or an amplitude of the stimulation signal transmitted by each element of the array.

20. The transcranial ultrasound system of claim 18, wherein transmitting the stimulation signal based on the plurality of skull aberration correction parameters comprises transmitting an additional pulse signal to counteract reflections at a surface of a skull indicated by the plurality of skull properties.

21. The transcranial ultrasound system of claim 18, wherein transmitting a stimulation signal based on the plurality of skull aberration correction parameters further comprises applying the first transcranial ultrasound array transducer or the second transcranial ultrasound transducer to a patient skull.

22. The transcranial ultrasound system of claim 18, wherein transmitting the stimulation signal based on the plurality of skull aberration correction parameters comprises transmitting long tone bursts based on the plurality of skull aberration correction parameters.

23. The transcranial ultrasound system of claim 18, wherein transmitting the stimulation signal based on the plurality of skull aberration correction parameters comprises applying a plurality of corrections to the stimulation signal using an inference neural network, wherein the plurality of skull aberration correction parameters are an input to the inference neural network.

24. A system, comprising:
a bone fragment generator configured to generate in-silico models of skull fragments; a processor; and
a compute server comprising an acoustic simulation engine and a neural network engine, wherein the compute server is configured to perform operations including:
generating, using the acoustic simulation engine, a plurality of simulated acoustic signals that are applied to the plurality of in-silico skull bone fragments;
capturing, using the acoustic simulation engine, a plurality of backscattered element responses to the plurality of simulated acoustic signals; and
generating, based on the plurality of backscattered responses, skull aberration correction parameters using the neural network engine for at least one element of a transcranial ultrasound transducer.

25. The system of claim 24, wherein the bone fragment generator generates in-silico skull bone fragments by varying at least one of a thickness, curvature, sound speed or density of the in-silico skull bone fragments.

26. The system of claim 24, wherein the neural network engine comprises a supervised learning system.

27. The system of claim 26, wherein the neural network engine comprises a pseudospectral simulator that simulates an effect of sound speed or density on a plurality of points of at least one of the plurality of in-silico bone fragments.

28. The system of claim 26, wherein the plurality of backscattered responses comprises pressure distributions on which the skull aberration correction parameters are based.

29. The system of claim 24, wherein the plurality of backscattered element responses comprises ultrasound signals scattered by the in-silico bone fragments to at least one receiver.

30. The system of claim 24, wherein the skull aberration parameters are further based on a transmitted pressure detected beyond the in-silico bone fragments.

31. The system of claim 24, wherein the neural network engine is trained by backpropagation on the plurality of backscattered responses.
